(19) United States
(12) Patent Application Publication
(10) Pub. No.: US 2002/0137954 A1
(43) Pub. Date: Sep. 26, 2002

(54) Process for preparing metal ascorbate and its precursor

(76) Inventors: Kouzou Kamiya, Hikari-shi (JP); Tetsuji Kaizu, Kudamatsu-shi (JP)

Correspondence Address: DAVIDSON,DAVIDSON & KAPPEL,LLC, 485 SEVENTH AVENUE,14TH FLOOR, NEW YORK, NY, 10018, US

(21) Appl. No.: 09/577,585

(22) Filed: May 25, 2000

(30) Foreign Application Priority Data
May 26, 1999 (JP) 146535/1999

(51) Int. Cl. C07D 307/62

(52) U.S. Cl. 549/477; 549/315

(57) ABSTRACT

A novel esterification process of 2-keto-L-gulonic acid is provided to obtain an improve degree of esterification. According to the process, 2-keto-L-gulonic acid anhydrous crystals are formed from its monohydrate crystals in water-soluble alcohol solvent. The anhydrous 2-keto-L-gulonic acid is esterified with lower alcohol to obtain a lower alkyl ester of 2-keto-L-gulonic acid. The resultant ester is lactonized to obtain a metal L-ascorbate.

### FIELD OF THE INVENTION

The present invention relates to a novel process for preparing a metal L-ascorbate. More specifically, in the process of the present invention, the precursor of the metal L-ascorbate of high quality is obtained in good yield with saving energy.

### BACKGROUND OF THE INVENTION

Usually, vitamin C (VC, L-ascorbic acid) is produced in an industrial scale by so-called Reichstein process (T. Reichstein, A. Grussner, Helv. Chim. Acta., 17, 311, 1994). In this process, as shown in Scheme 1, an alkyl ester of 2-keto-L-gulonic (2KGA) is obtained by esterification of diacetone-2-keto-L-gulonic acid (DAGA) with lower alcohol, for example, methanol. The yield of sodium L-ascorbate (ACS) is limited due to the esterification equilibrium. Then, the acetone formed is distilled away from a reaction mixture together with methanol to increase a degree of esterification. In this case, for the industrial production, a reaction mixture is heated for a long period of time, which causes a big problem of color in addition to those of the nature and purity of a product. Then, for increasing yield of the desired product, complicated production steps are required.

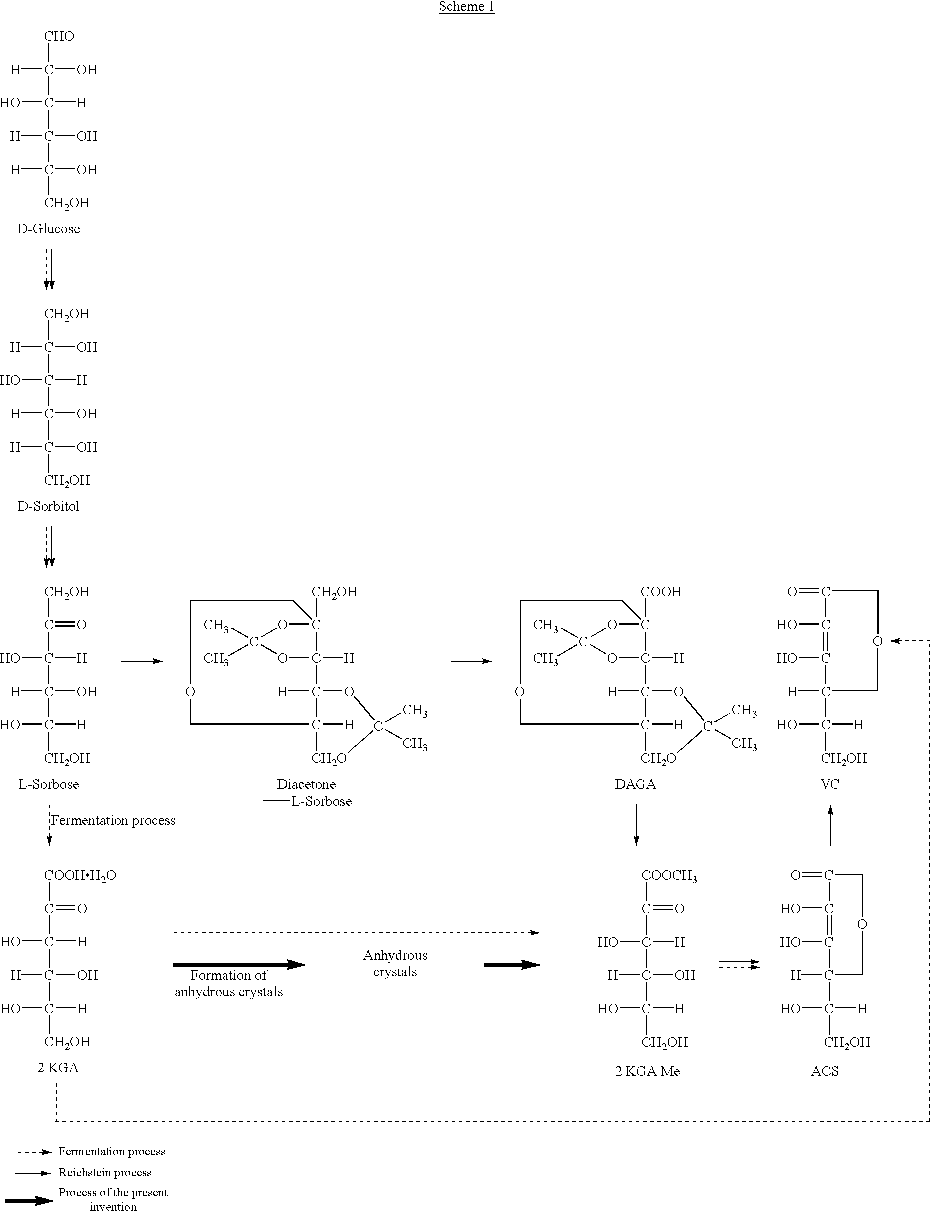

Recently, the fermentation process has been studied intensively and, at present, in particular, the technology for producing 2KGA from L-sorbose is put to practical use. This 2KGA is lactonized directly in the presence of an acid catalyst to obtain L-ascorbic acid. Alternatively, in a similar manner as DAGA in Reichstein process, it is lactonized through an ester and then converted into L-ascorbic acid. As known processes for improving esterification efficiency of 2KGE, for example, there are those described in JP-A 3-24068, JP-A 3-38579 and JP-A 7-206772. However, these processes involve difficult operations and, from the standpoint of the industrial production, they are hardly recognized to be economically advantageous processes.

In addition, 2-keto-L-gulonic acid crystals are obtained in the form of the monohydrate (2KGA.H₂O) from a fermentation mixture. Then, for its esterification, 2 molecules of water should be distilled away from a reaction mixture and this requires continuous addition of a large amount of lower alcohol to a reaction system to obtain a sufficient degree of esterification. For example, when 2KGA is esterified with lower alcohol, e.g., methanol, water should be distilled away from a reaction mixture together with methanol and, for the industrial production, it is necessary to add a large amount of methanol to a reaction system continuously (JP-A 5-194572). In this case, in a same manner as the esterification in Reichstain process as described above, a reaction mixture should be heated for a long period of time for the industrial production, which causes big problems of the nature, purity and color of a product. Then, for increasing yield of the desired product, complicated production steps are also required.

### OBJECTS OF THE INVENTION

Under these circumstances, an object of the present invention is to provide an esterification process of 2-keto-L-gulonic acid which can solve problems in conventional processes. That is, an object of the present invention is to provide an esterification process of 2-keto-L-gulonic acid, in which the esterification can be carried out efficiently with saving energy, in particular, with reducing the amount of lower alcohol to be used for the esterification, e.g., methanol (for example, with reducing both amounts of methanol to be distilled away and to be continuously added, largely), and with obtaining an improved degree of esterification.

This object as well as other objects and advantages of the present invention will become apparent to those skilled in the art from the following description.

### SUMMARY OF THE INVENTION

The present inventors have studied intensively to convert the raw material, 2KGA.H₂O into its anhydrous form. As a result, surprisingly, the present inventors have found that, when 2KGA.H₂O is added to water-soluble alcohol with stirring, anhydrous crystals of 2KGA are crystallized from the water-soluble alcohol after once 2KGA.H₂O has been dissolved therein. The present inventors have confirmed that 2KGA anhydrous crystals of highly improved quality can be obtained in good yield by filtrating off the anhydrous crystals thus formed.

The present inventors have further found that, in a process for preparing a metal L-ascorbate from this 2KGA anhydrous crystals through its lower alkyl ester, both amounts of methanol to be distilled away and to be continuously added can be reduced largely (about ¼ to ⅓ of the amount in a conventional process), and the desired product can be obtained in high yield. Thus the present invention has been completed.

That is, according to the present invention, there is provided:

(1) a process for preparing a lower alkyl ester of 2-keto-L-gulonic acid of the formula (I):

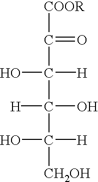

wherein R is a lower alkyl group, which comprises forming 2-keto-L-gulonic acid anhydrous crystals from its monohydrate crystals of the formula (II):

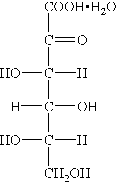

in water-soluble alcohol solvent, followed by a reaction with lower alcohol; and

(2) a process for preparing a metal L-ascorbate of the formula (III):

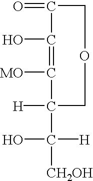

wherein M is alkali metal or alkaline earth metal, which comprises lactonizing the resultant ester.

### DETAILED DESCRIPTION OF THE INVENTION

2KGA monohydrate to be used as the raw material in the present invention can be obtained by crystallizing it from a concentrated fermentation mixture of microbial oxidation of L-sorbose according to a known method. Although the resultant 2KGA monohydrate contains 2 to 5% of adhesion water in addition to water of crystallization, it may be used in the form of moist crystals as it is without drying.

Examples of the water-soluble alcohol solvent to be used for crystallization of the anhydrous crystals in the present invention include lower alcohol having 1 to 4 carbon atoms such as methanol, ethanol, propanol, butanol and the like. Preferably, it is the same as the lower alcohol to be used for the esterification. For example, it is preferred to use methanol as the water-soluble alcohol solvent, when methyl ester of 2KGA is prepared.

The water-soluble alcohol solvent is preferably used in an amount 4 to 8 times molar quantity of 2KGA monohydrate. However, it can be used in an amount less than 4 times molar quantity of 2KGA monohydrate according to stirring conditions.

Formation of 2KGA anhydrous crystals can be carried out by addition of 2KGA monohydrate crystals to a water-soluble solvent warmed to 30 to 60° C. Normally, the conversion into the anhydrous crystals is completed by stirring at this temperature for 30 minutes to 2 hours. In order to increase yield of the anhydrous crystals, it is preferred to cool the crystallization mixture of the water-soluble alcohol solvent to 10° C. or lower after completion of formation of the anhydrous crystals, followed by aging for more than 1 hour and filtering off the resultant crystals. When the anhydrous crystals are filtered off, about 10% of 2KGA is transferred to the mother liquor. This can be recovered by distilling off the water-soluble alcohol solvent and combining the residue with the fermentation mixture, followed by concentration and crystallization.

The esterification reaction of 2KGA anhydrous crystals with lower alcohol can be carried out by using an acid as a catalyst according to the same manner as that in a conventional process using 2KGA. Examples of the acid include sulfuric acid, hydrochloric acid, phosphoric acid and the like. In particular, sulfuric acid is preferred. The acid can be used in an amount 0.0005 to 0.0020 time molar quantity, preferably 0.0007 to 0.0010 time molar quantity of 2KGA anhydrous crystals. Examples of the lower alcohol include that having 1 to 3 carbon atoms such as methanol, ethanol and the like. In particular, methanol is preferred.

The lower alcohol is used in an amount 4 to 7 times molar quantity of 2KGA anhydrous crystals in an initial stage of the esterification reaction. However, in order to increase yield of a lower alkyl ester of 2KGA, it is necessary to add lower alcohol continuously and distill off water formed together with lower alcohol.

When 2KGA anhydrous crystals is used according to the present invention, the required amount of lower alcohol to be added continuously in the esterification reaction is varied according to the water content of lower alcohol. However, it is about 20 times to 25 times molar quantity of 2KGA anhydrous crystals. On the other hand, in esterification of 2KGA.H₂O, it is necessary to add lower alcohol in an amount 70 times or more molar equivalent continuously to obtain the same yield as that in the present invention. Thus, in comparison with a conventional process, the desired metal L-ascorbate of high quality is prepared from 2KGA in good yield in the industrial production with remarkably saving energy according to the process of the present invention.

Lactonization of a lower alkyl ester of 2KGA thus obtained can be carried out according to a known method. For example, a solution of a hydroxide or bicarbonate of an alkali metal or an alkaline earth metal in methanol is added dropwise to a solution of the 2KGA lower alkyl ester in alcohol under reflux to lactonize the ester.

Examples of the metal salt of the objective product of the present invention, a metal ascorbate, include salts with an alkali metal such as sodium, potassium or the like and an alkaline earth metal such as calcium, magnesium or the like. The sodium salt is preferred.

The following examples further illustrate the present invention in detail. However, they are not to be construed to limit the scope thereof.

In the examples, color of the product was determined by measuring OD 430 nm with a spectrophotometer UV-160A (manufactured by Shimadzu Corporation).

### EXAMPLE 1

Dried 2-keto-gulonic acid monohydrate crystals (100 g, moisture content: 8.6%, purity: 90%) are added to methanol (100 ml) warmed to 40° C. and dissolved therein. After 5 to 10 minutes, 2-keto-L-gulonic acid anhydrous crystals started to crystallize. This temperature was kept for 30 minutes. Then, the mixture was cooled to 5° C. and the crystals were filtered off. The crystals were washed with methanol (15 ml). They were dried under reduced pressure to obtain 2-keto-L-gulonic acid anhydrous crystals (75.0 g, yield: 82%, purity: 99.0%, moisture content: 0.1%. color 430 nm. 20%: 0.002).

### EXAMPLE 2

Four portions of dried 2-keto-L-gulonic acid monohydrate crystals (total 100 g, moisture content: 8.6%, purity: 90%) were added to methanol (75 ml) warmed at 35° C. with stirring at 10 minutes intervals. After completion of the addition, the reaction was carried out at this temperature for 1 hour to form the corresponding anhydrous crystals. The reaction mixture was cooled to 5° C. and the crystals formed were filtered off. The crystals were washed with methanol (15 ml). They were dried under reduced pressure to obtain 2-keto-L-gulonic acid anhydrous crystals (82.4 g, yield: 90%, purity: 99.0%, moisture content: 0.1%, color 430 nm. 20%: 0.002).

### EXAMPLE 3

Four portions of undried 2-keto-L-gulonic acid monohydrate crystals (total 100 g, moisture content: 10.6%, purity: 88%) were added to methanol (75 ml) warmed at 35° C. with stirring at 10 minutes intervals. After completion of the addition, this temperature was kept for 30 minutes to form the corresponding anhydrous crystals. The reaction mixture was cooled to 10° C. and the crystals formed were filtered off. The crystals were washed with methanol (30 ml). They were dried under reduced pressure to obtain 2-keto-L-gulonic acid anhydrous crystals (80.6 g, yield: 88%, purity: 98.5%, moisture content: 0.5%, color 430 nm. 20%: 0.003).

### EXAMPLE 4

Methanol (150 ml, moisture content: 0.02%) was added to 2-keto-L-gulonic acid anhydrous crystals (150.0 g, moisture content: 0.1%, purity: 99.0%) and the mixture was warmed to form a solution.

After the temperature of the solution was elevated to its boiling point, conc. sulfuric acid (0.3 ml) was added thereto as a catalyst. To this was added methanol (600 ml) continuously over about 3 hours, while distilling away water formed together with methanol to keep the surface of the reaction mixture at a constant level. When a small amount of seed crystals was added after 30 minutes from initiation of esterification, methyl 2-keto-L-gulonate was crystallized out. After completion of addition of methanol, methanol (300 ml) was added to the mixture and crystals were dissolved under reflux.

Then, a 10.7 wt % solution of sodium hydroxide in methanol was added dropwise over about 2 hours under reflux to lactonize the ester. After 30 minutes from initiation of addition of the sodium hydroxide-methanol solution, sodium ascorbate started to crystallize out. When the mixture became pH 10, the addition of the sodium hydroxide-methanol solution was stopped and the mixture was cooled to room temperature (20° C.). Then, the crystals were filtered off. The crystals were washed with methanol (200 ml) and dried under reduced pressure to obtain sodium ascorbate (151.8 g, purity: 96.0%, yield 95.2%, color 430 nm. 58%: 0.08).

### EXAMPLE 5

Methanol (150 ml, moisture content: 0.02%) was added to 2-keto-L-gulonic acid anhydrous crystals (150.0 g, moisture content: 0.1%, purity: 99.0%) and the mixture was warmed to form a solution.

After the temperature of the solution was elevated to its boiling point, conc. sulfuric acid (0.3 ml) was added thereto as a catalyst. To this was added methanol (800 ml) continuously over about 4 hours, while distilling away water formed together with methanol to keep the surface of the reaction mixture at a constant level. When a small amount of seed crystals was added after 30 minutes from initiation of esterification, methyl 2-keto-L-gulonate was crystallized out. After completion of addition of methanol, methanol (300 ml) was added to the mixture and crystals were dissolved under reflux.

Then, a 10.7 wt % solution of sodium hydroxide in methanol was added dropwise over about 2 hours under reflux to lactonize the ester. After 30 minutes from initiation of addition of the sodium hydroxide-methanol solution, sodium ascorbate started to crystallize out. When the mixture became pH 10, the addition of the sodium hydroxide-methanol solution was stopped and the mixture was cooled to room temperature (20° C.). Then, the crystals were filtered off. The crystals were washed with methanol (200 ml) and dried under reduced pressure to obtain sodium ascorbate (151.5 g, purity: 96.6%, yield 95.6%, color 430 nm. 58%: 0.05).

### Comparative Example 1

Methanol (150 ml, moisture content: 0.26%) was added to 2-keto-L-gulonic acid monohydrate crystals (165.0 g, moisture content: 8.6%, purity: 90.0%) and the mixture was warmed to form a solution.

After the temperature of the solution was elevated to its boiling point, conc. sulfuric acid (0.3 ml) was added thereto as a catalyst. To this was added methanol (2000 ml) continuously over about 6 hours, while distilling away water formed together with methanol to keep the surface of the reaction mixture at a constant level. When a small amount of seed crystals was added after 30 minutes from initiation of esterification, methyl 2-keto-L-gulonate was crystallized out. After completion of addition of methanol, methanol (300 ml) was added to the mixture and crystals were dissolved under reflux.

Then, a 10.7 wt % solution of sodium hydroxide in methanol was added dropwise over about 2 hours under reflux to lactonize the ester. After 30 minutes from initiation of addition of the sodium hydroxide-methanol solution, sodium ascorbate started to crystallize out. When the mixture became pH 10, the addition of the sodium hydroxide-methanol solution was stopped and the mixture was cooled to room temperature (20° C.). Then, the crystals were filtered off. The crystals were washed with methanol (200 ml) and dried under reduced pressure to obtain sodium ascorbate (149.2 g, purity: 95.5%, yield 94.0%, color 430 nm. 58%: 0.97).

TABLE 1
Comparison of Examples 4 and 5 (present
invention) and Comparative Example 1 (conventional process)

| | | | | | Amount of |
|---|---|---|---|---|---|
| | | Sodium ascorbate | | | methanol |
| Example | Raw | Purity | Yield | OD430 | added |
| No. | material | (%) | (%) | nm | continuously |
| Example 4 | Anhydrous | 96.0 | 95.2 | 0.08 | 600 ml/150 g |
| | 2KGA | | | | |
| Example 5 | Anhydrous | 96.6 | 95.6 | 0.05 | 800 ml/150 g |
| | 2KGA | | | | |
| Comp. | 2KGA | 95.5 | 94.0 | 0.97 | 2000 ml/165 g* |
| Example 1 | mono- | | | | |
| | hydrate | | | | |

*corresponding to 150 g of anhydrous 2KGA

As is seen from Table 1, when 2KGA anhydrous crystals are used according to the present invention, the purity, yield and quality of the product, sodium L-ascorbate, are superior to those of the product of the conventional process. In addition, the reaction can be carried out with a very smaller amount of methanol to be added in comparison with the conventional process.

As described hereinabove, in a process for producing a metal ascorbate by using as a starting material 2-keto-L-gulonic acid through its alkyl ester, when 2-keto-L-gulonic acid monohydrate obtained from L-sorbitol by a conventional fermentation process is converted into its anhydrous crystals in a water-soluble alcohol solvent, followed by esterification according to the present invention, the desired product can be obtained efficiently with saving energy and without complicated reaction operations.

What is claimed is:

1. A process for preparing a lower alkyl ester of 2-keto-L-gulonic acid which comprises forming 2-keto-L-gulonic acid anhydrous crystals from its monohydrate crystals in water-soluble alcohol solvent, followed by a reaction with lower alcohol.

2. The process according to claim 1, wherein the lower alcohol is methanol.

3. The process according to claim 1, wherein the water-soluble alcohol solvent is methanol.

4. A process for preparing a metal L-ascorbate of the formula: 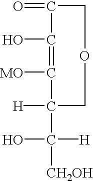
wherein M is alkali metal or alkaline earth metal, which comprises the steps of:
forming 2-keto-L-gulonic acid anhydrous crystals from its monohydrate crystals in water-soluble alcohol solvent, followed by a reaction with lower alcohol to obtain a lower alkyl ester of 2-keto-L-gulonic acid; and
lactonizing the resultant ester.

5. The process according to claim 4, wherein the lower alcohol is methanol.

6. The process according to claim 4, wherein the water-soluble alcohol solvent is methanol.

7. The process according to claim 4, wherein M is sodium.
